(19) United States
(12) Patent Application Publication
(10) Pub. No.: US 2020/0147404 A1
(43) Pub. Date: May 14, 2020

(54) SYSTEM AND METHOD FOR ENHANCING SENSORY FUNCTION

(71) Applicant: David Lewis Fried Oppenheim, Easthampton, MA (US)

(72) Inventor: David Lewis Fried Oppenheim, Easthampton, MA (US)

(21) Appl. No.: 16/562,836

(22) Filed: Sep. 6, 2019

Related U.S. Application Data
(60) Provisional application No. 62/760,891, filed on Nov. 13, 2018.

(51) Int. Cl. A61N 2/00; A61N 2/06; G06F 3/01

(52) U.S. Cl. CPC A61N 2/006; G06F 3/016; A61N 2/06

(57) ABSTRACT

A system and method for enhancing sensory function of an individual are disclosed. The system comprises a generalized brain-skin interface (GBSI), configured to enable the individual to experience senses. The system further comprises a sensor, a processor, and an epidermal neuro-oscillating patch (ENOP). The sensor is configured to receive environmental data surrounding the individual. The processor in communication with the sensor is configured to receive environmental data from the sensor and convert it into an analog vibration pattern. The epidermal neuro-oscillating patch (ENOP) is removably fastened to the skin of the individual, configured to produce a sequence of vibrations based on the analog vibration pattern received from the processor and directly transmit it to the skin for stimulating the nerves of the individual, thereby effectively training the brain of the individual to learn and equate the sequence of vibrations for experiencing senses of the physical environment of the individual.

### BACKGROUND OF THE INVENTION

A sense is a physiological capacity of organisms that provides data for perception. Sensory changes could affect the lifestyle of an individual and may cause them to face problems while communicating, enjoying activities, and staying involved with people. Sensory changes could lead to isolation. Sensory information is converted into nerve signals that are carried to the brain. There, the signals are turned into meaningful sensations. Traditional senses include sight (vision), hearing (audition), taste (gustation), smell (olfaction), and touch (somatosensation).

The human brain is capable of processing tremendous amounts of information in complex manners. The brain continuously receives and translates sensory information via nerves from multiple sensory sources including, for example, visual, auditory, olfactory, and tactile sources from the surrounding environment. The nerves are like wires, which send electrical impulses to the brain. The brain then makes sense of these impulses using contextual markers. For example, a blind individual learns to read by passing their fingers across a series of bumps. After a period of training, the brain does not need to consciously think of each letter to understand the text.

Many people and children are not able to experience the traditional senses such as sight (vision), hearing (audition), taste (gustation), smell (olfaction), and touch (somatosensation) due to pre-birth circumstances. Further, many individuals are not able to experience one ore more senses fully due to accidents, diseases and the bodies aging process. A certain amount of stimulation is required before the person becomes aware of a sensation. This minimum level of sensation is called a threshold.

Currently, devices such as glasses, hearing aids and cochlear implants can aid some individuals' ability to experience the traditional senses. The cochlear implant was developed to allow individuals with complete hearing loss to experience the hearing sense. The cochlear implant requires invasive surgery. The invasive surgery is not reversible. It can be painful and expensive. Also, a method developed for providing tactile feedback from artificial limbs requires an implant; an invasive, and expensive operation.

It has been demonstrated that some individuals who have lost their sight can train their brains to perceive their environment using echolocation by making a clicking sound with tongues. The brain can use the sonic signals to construct visual 3D representations of their environment. However, the conventional devices and methods for experiencing the traditional senses are expensive and are not efficient for individuals to train their brain. Individuals using the conventional devices and methods may feel discomfort and experience difficulty training their brains.

Therefore, there is a clear and present need for a system for sending electrical impulses to the human's brain to experience traditional senses such as sight (vision), hearing (audition), taste (gustation), smell (olfaction), and touch (somatosensation). Further, there is also a need to for an inexpensive and efficient system for individuals to train their brain for effectively experiencing the traditional senses.

### SUMMARY OF THE INVENTION

The present invention generally relates to a system and method for enhancing sensory function and more particularly relates to a system and method for sending electrical impulses to the human brain to experience senses such as, but not limited to, sight (vision), hearing (audition), taste (gustation), smell (olfaction), and touch (somatosensation).

In one embodiment, the system is configured to enable an individual to experience traditional senses such as sight (vision), hearing (audition), taste (gustation), smell (olfaction), and touch (somatosensation) by sending signals to the human's brain via nerves and the skin (epidermis). In one embodiment, the system is inexpensive and efficient for individuals to train their brain for effectively experiencing the senses. In one embodiment, the system comprises a generalized brain-skin interface (GBSI).

In one embodiment, the generalized brain-skin interface comprises one or more sensors, a processor, and one or more epidermal neuro-oscillating patches (ENOP) with a membrane. In one embodiment, the sensor is at least any one or a combination of, but not limited to, a camera, a microphone, tactile or touch sensors, surface sensors, a heat sensor, and a sonar sensor. The sensor is configured to receive data from the environment of the individual. In one embodiment, the sensor is configured to transmit environment data surrounding the individual to the processor. In another embodiment, the sensor is further configured to wirelessly transfer environmental data to the processor of the system. In one embodiment, the processor is in communication with at least any one or more sensors. In another embodiment, the processor is in wireless communication with at least one sensor. The processor is further configured to receive environmental data from at least one sensor and convert the received environmental data (digital information) into an analog vibration pattern.

In one embodiment, the epidermal neuro-oscillating patch (ENOP) is removably and adhesively fastened to the skin (epidermis) of the individual using an adhesive. In one embodiment, the epidermal neuro-oscillating patch (ENOP) is configured to receive the analog vibration pattern from the processor. In another embodiment, the epidermal neuro-oscillating patch (ENOP) is further configured to wirelessly receive the analog vibration pattern from the processor. In one embodiment, the epidermal neuro-oscillating patch is further configured to produce a sequence of vibrations based on the analog vibration pattern received from the processor. In one embodiment, the epidermal neuro-oscillating patch is further configured to directly transmit the sequence of vibrations to the skin for stimulating the nerves of the individual, thereby effectively training the brain to learn and equate the sequences of vibrations for experiencing senses of the environment surrounded by the individual. In one embodiment, the epidermal neuro-oscillating patch comprises one or more vibrating components. The one or more vibrating components of the epidermal neuro-oscillating patch are configured to produce periodic vibrations and non-periodic vibrations based on the analog vibration pattern received from the processor. In one embodiment, the one or more vibrating components of the epidermal neuro-oscillating patch comprise at least any one or a combination of a shape includes a circular, a rectangular, and a square-shaped structure.

In one embodiment, the senses are at least any one or a combination of, but not limited to, sight (vision), hearing (audition), taste (gustation), smell (olfaction), and touch (somatosensation). In one embodiment, the senses are at least any one or a combination of, but not limited to, temperature (thermoception/thermoreception), kinesthetic sense (proprioception), balance (equilibrioception), and internal stimuli. In one embodiment, the individual could sense salt and carbon dioxide (CO₂) in the blood, and also the sense of hunger and thirst. These could be used in the treatment of various medical conditions, and sensory augmentation in law enforcement, firefighting, and entertainment events.

In one embodiment, the system could use at least 2 epidermal neuro-oscillating patches fastened to or placed against the skin of the individual at different locations, for example, a neck portion and a lower back portion or an armpit region. In one embodiment, the system is further configured to enable the individual to train the brain for immersion perception of, but not limited to, three-dimensional (3D) audios and visuals by transmitting a sequence of vibrations generated by one or more epidermal neuro-oscillating patches (ENOP) to the skin for stimulating the nerves of the individual. In one embodiment, the individual could sense, but not limited to, proximity to heat or other forms of radiation using the system. The dual epidermal neuro-oscillating patches produce a sequence of vibrations or oscillations based on the received offset visual images or sound inputs from the processor in the form of analog vibration pattern.

In one embodiment, the system is configured to enable the individual to experience the traditional senses, such as, but not limited to, sight (vision), hearing (audition), taste (gustation), smell (olfaction), and touch (somatosensation). In one embodiment, the individuals could sense hearing (audition) who are suffering from a hearing loss. In one embodiment, the system is configured to receive pulsations of modulating frequency and directly transmit to the skin of the individual in a form of a sequence of vibrations using the epidermal neuro-oscillating patch for experiencing a wide range of sonic inputs. The individual could also sense ultrasonic and subsonic tones by transmitting a sequence of vibrations to the skin via the epidermal neuro-oscillating patch. In one embodiment, the individuals could sense cutaneous touch and limb motion who have suffered limb loss or spinal trauma using tactile sensors with the system. In one embodiment, the system is further configured to enable the individual to feel the artificial (prosthetic) limb as a genuine part of their body using surface sensors. The individual could sense the position and movement of the artificial (prosthetic) limb using the system with one or more surface sensors. In one embodiment, the individual is at least any one of, but not limited to, a person or a child with disabilities include, the visually impaired, hearing impaired, amputees, and an impaired sense of taste and smell.

In one embodiment, a method for enhancing sensory function is disclosed. At one step, one or more sensors receive environmental data surrounding the individual and transmit that data to the processor of the system. At another step, the processor converts the received environmental data into an analog vibration pattern and transmits it to the epidermal neuro-oscillating patch of the system. Further, at another step, the epidermal neuro-oscillating patch produces a sequence of vibrations based on the analog vibration pattern received from the processor and directly transmits it to the skin for stimulating the nerves of the individual to train the brain to equate the sequence of vibrations for experiencing senses of the environment surrounded by the individual.

One aspect of the present disclosure is directed to a system for enhancing sensory function, comprising: a) a generalized brain-skin interface (GBSI) configured to enable an individual to experience senses, comprising i) one or more sensors configured to receive environmental data surrounding an individual; ii) a processor in communication with the at least one or more sensors, wherein the processor is configured to receive environmental data from at least one or more sensors and convert the received environmental data into an analog vibration pattern, and iii) one or more epidermal neuro-oscillating patches (ENOP) with a membrane removably fastened to the skin of the individual, wherein each epidermal neuro-oscillating patch is configured to produce a sequence of vibrations based on the analog vibration pattern received from the processor and directly transmit to the skin for stimulating the nerves of the individual, thereby effectively training the brain of the individual to learn and equate the sequence of vibrations for experiencing senses of a physical environment surrounded by the individual.

In one embodiment, the one or more sensors are at least any one or a combination of a camera, a microphone, tactile or touch sensors, surface sensors, a heat sensor, and a sonar sensor. In another embodiment, the one or more sensors are further configured to wirelessly transfer environment data to the processor of the system. In one embodiment, the senses at least any one or a combination of sight (vision), hearing (audition), taste (gustation), smell (olfaction), and touch (somatosensation). In another embodiment, the senses at least any one or a combination of temperature (thermoception), kinesthetic sense (proprioception), balance (equilibrioception), and internal stimuli. In one embodiment, at least one epidermal neuro-oscillating patch (ENOP) is adhesively fastened to a neck portion of the individual.

In another embodiment, at least one epidermal neuro-oscillating patch (ENOP) is adhesively fastened to a lower back portion or an armpit region of the individual. In one embodiment, one or more epidermal neuro-oscillating patches (ENOP) are further configured to wirelessly receive the analog vibration pattern from the processor. In another embodiment, the one or more epidermal neuro-oscillating patches (ENOP) comprise one or more vibrating components. In a related embodiment, the one or more vibrating components of each epidermal neuro-oscillating patch are configured to produce periodic vibrations and non-periodic vibrations based on the analog vibration pattern received from the processor, wherein the one or more vibrating components of each epidermal neuro-oscillating patch (ENOP) comprise at least any one or a combination of a shape includes a circular, a rectangular, and a square-shaped structure. In one embodiment, the system is further configured to enable the individual to train the brain for immersion perception of three-dimensional (3D) audios and visuals by transmitting a sequence of vibrations generated by the one or more epidermal neuro-oscillating patches (ENOP) to the skin for stimulating the nerves of the individual.

Another aspect of the present disclosure is directed to a system for enhancing sensory function, comprising: (a) a generalized brain-skin interface (GBSI) configured to enable an individual to experience senses, comprising (i) one or more sensors configured to receive environment data surrounding by an individual; (ii) a processor in wireless communication with the at least one or more sensors, wherein the processor is configured to wirelessly receive environmental data from at least one or more sensors and convert environmental data into an analog vibration pattern, and (iii) one or more epidermal neuro-oscillating patches (ENOP) with a membrane removably fastened to the skin of the individual, wherein each epidermal neuro-oscillating patch comprising one or more vibrating components, configured to produce a sequence of vibrations based on the analog vibration pattern received from the processor and directly transmit to the skin for stimulating the nerves of the individual, thereby effectively training the brain of the individual to learn and equate the sequence of vibrations for experiencing senses of the physical environment of the individual.

Another aspect of the present disclosure is directed to a method for enhancing sensory function, comprises: a) receiving environment data using one or more sensors and transmitting to a processor; b) converting the received environment data into an analog vibration pattern by the processor; c) transmitting the analog vibration pattern to an epidermal neuro-oscillating patch (ENOP) from the processor, and d) producing a sequence of vibrations by the epidermal neuro-oscillating patch and directly transmitting to the skin for stimulating the nerves of the individual to train the brain for learning and to equate the sequence of vibrations for experiencing senses of a physical environment of an individual.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 illustrates a block diagram of a system for enhancing sensory function according to an embodiment of the present invention;

FIG. 2 shows a perspective view of an epidermal neuro-oscillating patch (ENOP) fastened to skin at a neck portion of an individual according to one embodiment of the present invention;

FIG. 3 shows a perspective view of a sensor, for example, a camera securely positioned at a head portion of the individual according to one embodiment of the present invention;

FIG. 4 illustrates a perspective view of the individual experiences a sense of sight (vision) using the epidermal neuro-oscillating patch of the system according to one embodiment of the present invention;

FIG. 5 shows a top portion of the epidermal neuro-oscillating patch with a plurality of circular vibrating components according to one embodiment of the present invention;

FIG. 6 shows a top portion of the epidermal neuro-oscillating patch with a plurality of vibrating strip components according to another embodiment of the present invention;

FIG. 7 shows a top portion of the epidermal neuro-oscillating patch with a plurality of rectangular vibrating components according to another embodiment of the present invention;

FIG. 8 shows a top view of the epidermal neuro-oscillating patch of the system according to one embodiment of the present invention;

FIG. 9 shows a side view of the epidermal neuro-oscillating patch of the system according to one embodiment of the present invention; and

FIG. 10 shows a flowchart of a method for enhancing sensory function according to one embodiment of the present invention.

### DETAILED DESCRIPTION

The present invention generally relates to a system and method for enhancing sensory function and more particularly relates to a system and method for sending information to a human's brain to experience senses such as, but not limited to, sight (vision), hearing (audition), taste (gustation), smell (olfaction), and touch (somatosensation).

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

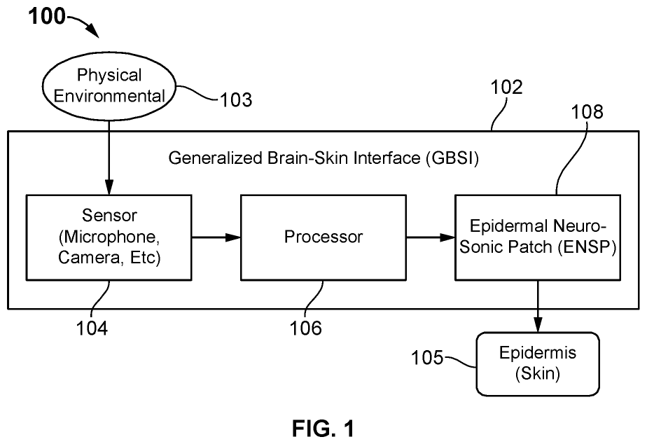

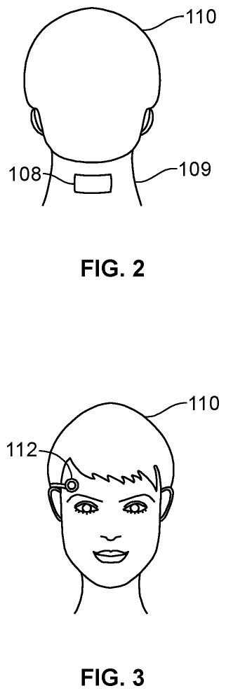

Referring to FIG. 1, a system 100 for sending electrical impulses to the human brain for experiencing senses is disclosed. In one embodiment, the system 100 is configured to enable an individual 110 (shown in FIG. 2) to experience traditional senses such as sight (vision), hearing (audition), taste (gustation), smell (olfaction), and touch (somatosensation) by sending electrical impulses to the human's brain via nerves and epidermis (skin) 105. In one embodiment, the system 100 is inexpensive and efficient for individuals to train their brain for effectively experiencing the senses. In one embodiment, the system 100 comprises a generalized brain-skin interface (GBSI) 102.

In one embodiment, the generalized brain-skin interface 102 comprises one or more sensors 104, a processor 106, and one or more epidermal neuro-oscillating patches (ENOP) 108 with a membrane. In one embodiment, the sensor 104 is at least any one or a combination of, but not limited to, a camera, a microphone, tactile or touch sensors, surface sensors, a heat sensor, and a sonar sensor. The sensor 104 is configured to receive environmental data from a physical environment 103 surrounding the individual 110. In one embodiment, the sensor 104 is configured to transmit environmental data surrounding the individual 110 to the processor 106. In another embodiment, the sensor 104 is further configured to wirelessly transfer environment data to the processor 106 of the system 100. In one embodiment, the processor 106 is in communication with at least any one or more sensors 104. In another embodiment, the processor 106 is in wireless communication with at least one sensor 104. The processor 106 is further configured to receive environmental data from at least one sensor 104 and convert received environmental data (digital information) into an analog vibration pattern. In one embodiment, the processor 106 of the system 100 could be positioned anywhere.

In one embodiment, the epidermal neuro-oscillating patch (ENOP) 108 is removably and adhesively fastened to the skin (epidermis) 105 of the individual 110 using an adhesive. In one embodiment, the epidermal neuro-oscillating patch (ENOP) 108 is configured to receive the analog vibration pattern from the processor 106. In another embodiment, the epidermal neuro-oscillating patch (ENOP) 108 is further configured to wirelessly receive the analog vibration pattern from the processor 106. In one embodiment, the epidermal neuro-oscillating patch 108 is further configured to produce a sequence of vibrations based on the analog vibration pattern received from the processor 106.

The epidermal neuro-oscillating patches 108 may further be configured to directly transmit the sequence of vibrations to the skin 105 for stimulating the nerves of the individual 110, thereby effectively training the brain to learn and equate the sequences of vibrations for experiencing senses of the physical environment 103 surrounded the individual 110. In one embodiment, the senses are at least any one or a combination of, but not limited to, sight (vision), hearing (audition), taste (gustation), smell (olfaction), and touch (somatosensation). In one embodiment, the senses are at least any one or a combination of, but not limited to, temperature (thermoception/thermoreception), kinesthetic sense (proprioception), balance (equilibrioception), and internal stimuli. In one embodiment, the individual 110 could sense salt and carbon dioxide (CO₂) in the blood, and also the sense of hunger and thirst. These could be used in the treatment of various medical conditions, and sensory augmentation in law enforcement, firefighting, and entertainment events.

The system 100 could use at least 2 epidermal neuro-oscillating patches 108 fastened to the skin 105 of the individual 110 at different locations, for example, a neck portion 109 (shown in FIG. 2) and a lower back portion or an armpit region. In one embodiment, the system 100 is further configured to enable the individual 110 to train the brain for immersion perception of, but not limited to, three-dimensional (3D) audios and visuals by transmitting a sequence of vibrations generated by one or more epidermal neuro-oscillating patches (ENOP) 108 to the skin 105 for stimulating the nerves of the individual 110. In one embodiment, the individual 110 could sense proximity to heat or other forms of radiation using the system 100. The dual epidermal neuro-oscillating patches 108 produce a sequence of vibrations or oscillations based on the received offset visual images, audio signals or other inputs from the processor 106 in the form of analog vibration pattern.

In one embodiment, the system 100 is configured to enable the individual 110 to experience the traditional senses, such as, but not limited to, sight (vision), hearing (audition), taste (gustation), smell (olfaction), and touch (somatosensation). In one embodiment, individuals who are suffering from a hearing loss could sense audio input (audition). In one embodiment, the system 100 is configured to receive pulsation of modulating frequency and directly transmits to the skin 105 of the individual in a form of a sequence of vibrations using the epidermal neuro-oscillating patch 108 for experiencing a wide range of sonic inputs.

The individual 110 could also sense ultrasonic and subsonic tones by transmitting a sequence of vibrations to the skin 105 via the epidermal neuro-oscillating patch 108. In one embodiment, the individuals could sense cutaneous touch and limb motion who have suffered limb loss, neural damage or spinal trauma using tactile sensors with the system 100. In one embodiment, the system 100 is further configured to enable the individual 110 to feel the artificial (prosthetic) limb as a genuine part of their body using surface sensors. The individual 110 could sense the position and movement of the artificial (prosthetic) limb using the system 100 with one or more surface or sub-surface sensors. In one embodiment, the individual 110 is at least any one of, but not limited to, a person or a child with disabilities including the visually impaired, hearing impaired, amputees, and an impaired sense of taste and smell.

Referring to FIG. 2, the epidermal neuro-oscillating patch 108 is securely affixed to a neck portion 109 of the individual 110 is disclosed. In one embodiment, at least one epidermal neuro-oscillating patch 108 is removably and adhesively fastened to the skin 105 (shown in FIG. 1) at, but not limited to, the neck portion 109 of the individual 110. The epidermal neuro-oscillating patch 108 is directly fastened to a collection of nerve endings in any particular section of the individual's skin 105. In some embodiments, one or more epidermal neuro-oscillating patches 108 are removably fastened to the skin 105 at, but not limited to, a lower back portion and an armpit region of the individual 110. In one embodiment, the epidermal neuro-oscillating patch 108 is configured to produce a sequence of oscillations that stimulate the dermal nerves of the individual 110.

Referring to FIG. 3, a visual sensor 112 is securely positioned at the head portion of the individual 110 is disclosed. In one embodiment, at least one visual sensor 112 for example, a camera, is securely positioned, but not limited to, a head portion, a chest, arms, and a back side of the individual 110. In an examplary embodiment, the visual sensor 112 is positioned to the head facing forward so that the visual information presented changes based on the direction the individual 110 turns their head. In one embodiment, the visual sensor 112 is configured to capture environmental data surrounding the individual 110 and transfer the captured environmental data to the processor 106 of the system 100. In another embodiment, the visual sensor 112 is further configured to wirelessly transfer the captured environmental data to the processor 106 of the system 100.

The individual 110 could train their brain easier when additional information can be used in conjunction with the information provided by the generalized brain-skin interface 102 of the system 100. For instance, feeling an object that one is holding in front of one's face as a visual sensor sends information about that object will more quickly draw neural connections about the information presented. The environmental data includes, but is not limited to, a shape of an object or a product, distance from an object, color, thermal images/signatures, and ultra-violet and infrared photography.

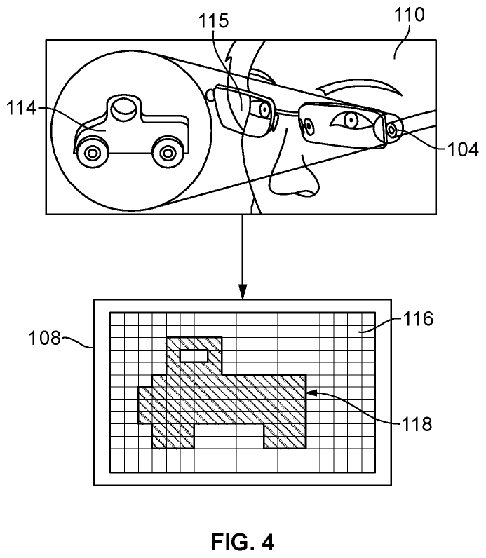

Referring to FIG. 4, the individual 110 could experience the sense of sight (vision) using the system 100 is disclosed. In one embodiment, a sensor 104, for example, a camera, is securely positioned on, but not limited to, spectacles 115 for capturing the physical environment 103 (shown in FIG. 1) surrounding the individual 110. In one embodiment, the sensor 104 is configured to wirelessly transfer the captured environmental data or sensory data, for example, a car 114, to the processor 106 of the system 100. The processor 106 is configured to convert the captured environmental data, for example, a car 114, into an analog vibration pattern.

The epidermal neuro-oscillating patch 108 of the system 100 is configured to produce a sequence of vibrations/oscillations 118 via the one or more vibrating components 116 based on the analog vibration pattern received from the processor 106 and directly transmit to the sequence of vibrations 118 in real-time to the skin 105 for stimulating the nerves of the individual 110, thereby effectively training the brain to learn and equate the sequences of vibrations 118 for experiencing the sense of sight (vision) of environmental data, for example, a car 114.

The vibrating components 116 of each epidermal neuro-oscillating patch (ENOP) 108 may comprise at least any one or a combination of shapes including, but not limited to, a circular, a rectangular, and a square-shaped structure. In one embodiment, the vibrating components 116 of each epidermal neuro-oscillating patch 108 are configured to produce periodic vibrations and non-periodic vibrations at varying frequencies based on the analog vibration pattern, which is received from the processor 106.

Direct stimulation of the nerve endings in a section of the skin 105 through a process of training teaches the brain to recognize those sequences of vibrations/oscillations 118 as a source of environmental data. Much like the fingers of a blind person can transmit information from multiple contact points along the skin 105 as they move their hands across a line of Braille. The greater the number of points, the more information that can be transmitted.

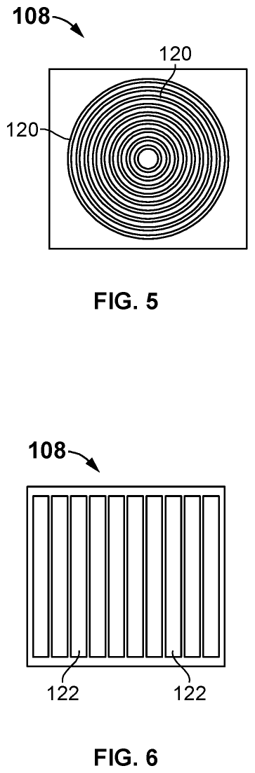

Referring to FIG. 5, the epidermal neuro-oscillating patch 108 of the system 100 is disclosed. In one embodiment, the epidermal neuro-oscillating patch 108 comprises one or more circular/ring vibrating components 120. The circular vibrating components 120 are configured to produce a sequence of vibrations or oscillations based on the analog vibration pattern received from the processor 106 (shown in FIG. 1) of the system 100. In some embodiments, the circular vibrating components 120 are configured to produce periodic vibrations and non-periodic vibrations based on environment data captured from the one or more sensors 104.

Referring to FIG. 6, the epidermal neuro-oscillating patch 108 of the system 100 is disclosed. In another embodiment, the epidermal neuro-oscillating patch 108 comprises one or more vibrating strip components 122. The vibrating strip components 122 are configured to produce a sequence of vibrations or oscillations based on the analog vibration pattern received from the processor 106 of the system 100. In some embodiments, the vibrating strip components 120 are configured to produce periodic vibrations and non-periodic vibrations based on environmental data captured from one or more sensors 104, for example, a camera or a tactile sensor.

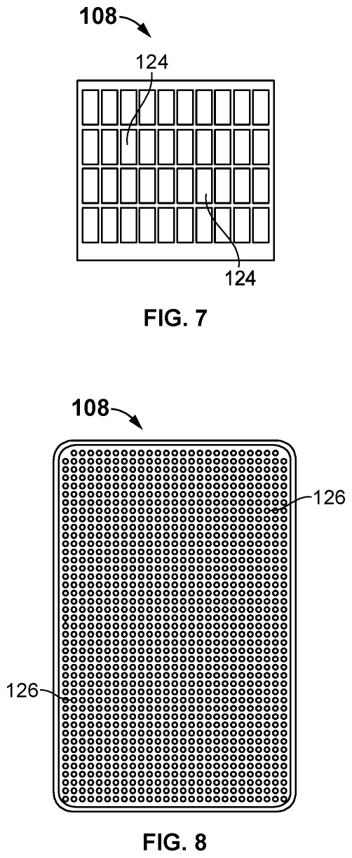

Referring to FIG. 7, the epidermal neuro-oscillating patch 108 of the system 100 is disclosed. In another embodiment, the epidermal neuro-oscillating patch 108 comprises one or more rectangular or square vibrating components 124. The rectangular or square vibrating components 124 are configured to produce a sequence of vibrations or oscillations based on the analog vibration pattern received from the processor 106 of the system 100. In some embodiments, the rectangular or square vibrating components 124 are configured to produce periodic vibrations and non-periodic vibrations based on environmental data captured from one or more sensors 104, for example, a camera or a tactile sensor.

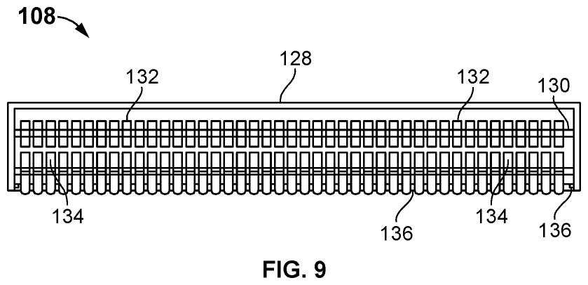

Referring to FIGS. 8-9, an epidermal neuro-oscillating patch 108 of the system 100 is disclosed. In one embodiment, the epidermal neuro-oscillating patch 108 comprises a plurality of pins or contact points 126. In one embodiment, the plurality of pins 126 are arranged in, but not limited to, a column configuration, a row configuration, and any other combination thereof. In one embodiment, the number of the plurality of pins 126 of the epidermal neuro-oscillating patch 108 is about, but not limited to, 1202. In one embodiment, each pin 126 has a top diameter of about, but not limited to, 1.25 mm and a bottom diameter of about, but not limited to, 1.5 mm. In one embodiment, each pin 126 has a height of about, but not limited to, 3.00 mm and are separated from each other with a distance or a space of about, but not limited to, 0.75 mm. In one embodiment, the plurality of pins 126 are made of a material, but not limited to, aluminum.

In one embodiment, the epidermal neuro-oscillating patch 108 of the system 100 has a width of about, but not limited to, 57.7022 mm and a height of about, but not limited to, 90.0875 mm. In one embodiment, the epidermal neuro-oscillating patch 108 of the system 100 has a depth of about, but not limited to, 14.00 mm. In one embodiment, the epidermal neuro-oscillating patch 108 further comprises a casing 128, a holder 130, a plurality of electromagnets 132, a plurality of permanent magnets 134, and a bottom casing 136. In one embodiment, the holder 130 is configured to securely hold the plurality of electromagnets 132. In one embodiment, each permanent magnet 134 has a height of about, but not limited to, 3.00 mm and a diameter of about 1.25 mm. In one embodiment, the permanent magnets 134 are made from, but not limited to, N52 neodymium (nickel-plated).

In one embodiment, each electromagnet 132 has a height of about, but not limited to, 4.00 mm and a diameter of about, but not limited to, 1.25 mm. In one embodiment, the operating voltage is about, but not limited to, 3.6 v and the operating current is about, but not limited to, 20 A. In one embodiment, a copper wire is used to electrically connected to a power source. In one embodiment, the power source is, but not limited to, a Li-Ion battery with a capacity of 3000 mAh. In one embodiment, the nominal voltage of the battery is about, but not limited to, 3.6 V and continuous current is about, but not limited to, 20 A. The maximum current of the battery is about, but not limited to, 40 A and cycle life is about, but not limited to, 600 times. In one embodiment, the size of the battery is about, but not limited to, 18.5 mm. In one embodiment, the weight of the battery is about, but not limited to, 65g.

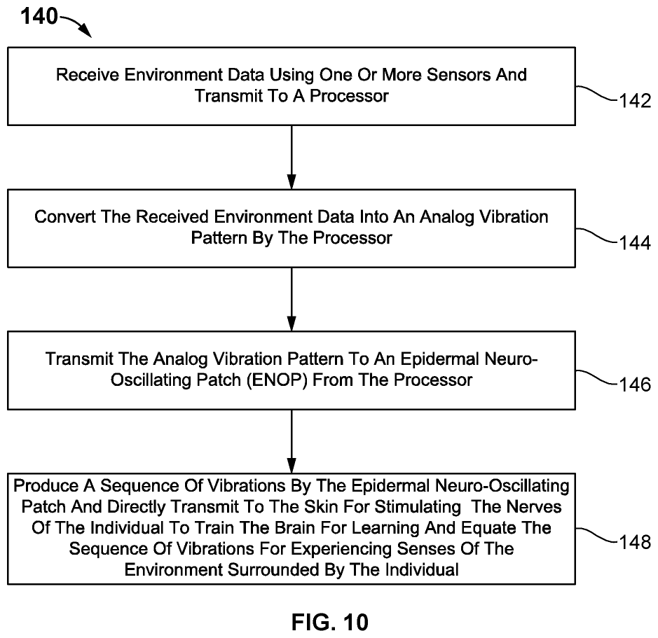

Referring to FIG. 10, a method 140 for enhancing sensory function is disclosed. At step 142, one or more sensors 104 receive environmental data surrounding the individual 110 and transmit it to the processor 106 of the system 100. At steps 144 and 146, the processor 106 converts the received environmental data into an analog vibration pattern and transmits it to the epidermal neuro-oscillating patch 108 of the system 100. Further, at step 148, the epidermal neuro-oscillating patch 108 produces a sequence of vibrations based on the analog vibration pattern received from the processor 106 and directly transmits it to the skin 105 for stimulating the nerves of the individual 110 to train the brain for learning and equate the sequence of vibrations for experiencing senses of the physical environment 103 surrounding the individual 110.

In one embodiment, the system 100 comprises a global positioning system (GPS). The system 100 could enable the individual 110 to know where they are intuitively concerning other fixed elements using GPS data. In one embodiment, the system 100 could enable the individual 110 to see in the dark and all directions simultaneously using the sensor 104, for example, a sonar sensor. In one embodiment, system 100 is further configured to enable two or more individuals to directly communicate via virtual communications without using any additional electronic devices, for example, a smartphone or a screen.

Virtual environment sense equivalency to create a sense of presence through simulated tactile feedback inside of virtual reality experience. In one embodiment, the system 100 is further configured to enable the individuals to directly connect to the internet without using a screen or an audio input. In one embodiment, the system 100 is further configured to provide heat vision for the individual 110, which is useful in environments of dense air-borne particulate and thermal extremes such as encountered in fire fighting situations. In one embodiment, the system 100 could be used with one or more radiation sensors, which are used for emergency services. In one embodiment, the system 100 is further configured to aid musicians to tune ears for perfect pitch.

The advantages of the present invention include: the system 100 provides neural stimuli in a non-invasive, non-surgical and inexpensive manner. The system 100 could be used across any number of different sensory inputs without any effect and danger for the individuals. The system 100 provides comfort for the individuals and is inexpensive, simple in design, easy to use, reliable, and safe for the individuals. In one embodiment, the system 100 could provide complex environmental information via a sequence of vibrations and patterns provided by one or more vibrating components of the epidermal neuro-oscillating patch 108.

The senses may be, without limitation, one of or a combination of sight (vision), hearing (audition), taste (gustation), smell (olfaction), touch (somatosensation), temperature (thermoception), kinesthetic sense (proprioception), balance (equilibrioception), and internal stimuli. In one example, the one or more epidermal neuro-oscillating patches (ENOP) are further configured to wirelessly receive the analog vibration pattern from the processor. The one or more vibrating components of each epidermal neuro-oscillating patch may be configured to produce periodic vibrations and non-periodic vibrations based on the analog vibration pattern received from the processor. The one or more vibrating components of each epidermal neuro-oscillating patch (ENOP) may comprise at least any one or a combination of a shape including a circular, a rectangular, and a square-shaped structure. In one example, the system is further configured to enable the individual to train the brain for immersion perception of three-dimensional (3D) audios and visuals by transmitting a sequence of vibrations generated by the one or more epidermal neuro-oscillating patches (ENOP) to the skin for stimulating the nerves of the individual.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions.

Although specific terms may be employed herein, they are used only in generic and descriptive sense and not for purposes of limitation. Accordingly, the present invention is not limited to the specific embodiments illustrated herein. While the above is a complete description of the preferred embodiments of the invention, various alternatives, modifications, and equivalents may be used. Therefore, the above description and the examples should not be taken as limiting the scope of the invention, which is defined by the appended claims.

1. A system for enhancing sensory function, comprising:
a generalized brain-skin interface (GBSI) configured to enable an individual to experience senses, comprising
one or more sensors configured to receive environment data surrounding by an individual;
a processor in communication with the at least one or more sensors, wherein the processor is configured to receive environment data from at least one or more sensors and convert the received environmental data into an analog vibration pattern, and
one or more epidermal neuro-oscillating patches (ENOP) with or without a membrane removably fastened to the skin of the individual, wherein each epidermal neuro-oscillating patch is configured to produce a sequence of vibrations based on the analog vibration pattern received from the processor and directly transmit to the skin for stimulating the nerves of the individual, thereby effectively training the brain of the individual to learn and equate the sequence of vibrations for experiencing senses of a physical environment surrounded by the individual.

2. The system of claim 1, wherein the one or more sensors are at least any one or a combination of a camera, a microphone, tactile or touch sensors, surface sensors, a heat sensor, and a sonar sensor.

3. The system of claim 1, wherein the one or more sensors are further configured to wirelessly transfer environment data to the processor of the system.

4. The system of claim 1, wherein the senses at least any one or a combination of sight (vision), hearing (audition), taste (gustation), smell (olfaction), and touch (somatosensation).

5. The system of claim 1, wherein the senses at least any one or a combination of temperature (thermoception), kinesthetic sense (proprioception), balance (equilibrioception), and internal stimuli.

6. The system of claim 1, wherein at least one epidermal neuro-oscillating patch (ENOP) is adhesively fastened to a neck portion of the individual.

7. The system of claim 1, wherein at least one epidermal neuro-oscillating patch (ENOP) is adhesively fastened to a lower back portion or an armpit region of the individual.

8. The system of claim 1, wherein the one or more epidermal neuro-oscillating patches (ENOP) are further configured to wirelessly receive the analog vibration pattern from the processor.

9. The system of claim 1, wherein the one or more epidermal neuro-oscillating patches (ENOP) comprise one or more vibrating components.

10. The system of claim 9, wherein the one or more vibrating components of each epidermal neuro-oscillating patch are configured to produce periodic vibrations and non-periodic vibrations based on the analog vibration pattern received from the processor, wherein the one or more vibrating components of each epidermal neuro-oscillating patch (ENOP) comprise at least any one or a combination of a shape includes a circular, a rectangular, and a square-shaped structure.

11. The system of claim 1, is further configured to enable the individual to train the brain for immersion perception of three-dimensional (3D) audios and visuals by transmitting a sequence of vibrations generated by the one or more epidermal neuro-oscillating patches (ENOP) to the skin for stimulating the nerves of the individual.

12. A system for enhancing sensory function, comprising:
a generalized brain-skin interface (GBSI) configured to enable an individual to experience senses, comprising
one or more sensors configured to receive environment data surrounding by an individual;
a processor in wireless communication with the at least one or more sensors, wherein the processor is configured to wirelessly receive environmental data from at least one or more sensors and convert environmental data into an analog vibration pattern, and
one or more epidermal neuro-oscillating patches (ENOP) with or without a membrane removably fastened to the skin of the individual, wherein each epidermal neuro-oscillating patch comprises one or more vibrating components, configured to produce a sequence of vibrations based on the analog vibration pattern received from the processor and directly transmit to the skin for stimulating the nerves of the individual, thereby effectively training the brain of the individual to learn and equate the sequence of vibrations for experiencing senses of a physical environment surrounding the individual.

13. The system of claim 12, wherein the one or more sensors are at least any one or a combination of a camera, a microphone, tactile or touch sensors, surface sensors, a heat sensor, and a sonar sensor.

14. The system of claim 12, wherein the senses are at least any one or a combination of sight (vision), hearing (audition), taste (gustation), smell (olfaction), touch (somatosensation), temperature (thermoception), kinesthetic sense (proprioception), balance (equilibrioception), and internal stimuli.

15. The system of claim 12, wherein the one or more epidermal neuro-oscillating patches (ENOP) are further configured to wirelessly receive the analog vibration pattern from the processor.

16. The system of claim 12, wherein at least one epidermal neuro-oscillating patch (ENOP) is adhesively fastened to a neck portion, a lower back portion or an armpit region of the individual.

17. The system of claim 12, wherein the one or more vibrating components of each epidermal neuro-oscillating patch are configured to produce periodic vibrations and non-periodic vibrations based on the analog vibration pattern received from the processor.

18. The system of claim 12, wherein the one or more vibrating components of each epidermal neuro-oscillating patch (ENOP) comprise at least any one or a combination of a shape includes a circular, a rectangular, and a square-shaped structure.

19. The system of claim 12, is further configured to enable the individual to train the brain for immersion perception of three-dimensional (3D) audios and visuals by transmitting a sequence of vibrations generated by the one or more epidermal neuro-oscillating patches (ENOP) to the skin for stimulating the nerves of the individual.

20. A method for enhancing sensory function, comprises:
receiving environmental data using one or more sensors and transmitting to a processor;
converting the received environmental data into an analog vibration pattern by the processor;
transmitting the analog vibration pattern to an epidermal neuro-oscillating patch (ENOP) from the processor, and
producing a sequence of vibrations by the epidermal neuro-oscillating patch and directly transmitting to the skin for stimulating the nerves of the individual to train the brain for learning and equate the sequence of vibrations for experiencing senses of a physical environment surrounded by the individual.
